(19) United States
(12) Patent Application Publication
(10) Pub. No.: US 2006/0036262 A1
(43) Pub. Date: Feb. 16, 2006

(54) Loading device for loading a lens folding cartridge with an intraocular lens, lens folding cartridge and set for implantation

(75) Inventor: Emil Hohl, Heerbrugg (CH)

Correspondence Address: NIXON & VANDERHYE, PC, 901 NORTH GLEBE ROAD, 11TH FLOOR, ARLINGTON, VA, 22203, US

(73) Assignee: Medicel AG, Widnau (CH)

(21) Appl. No.: 11/202,028

(22) Filed: Aug. 12, 2005

(30) Foreign Application Priority Data
Aug. 12, 2004 (CH) CH 01334/04

(51) Int. Cl. A61F 9/00

(52) U.S. Cl. 606/107

(57) ABSTRACT

The loading device to load a lens folding cartridge with an intraocular lens comprises a support with a seat to receive the still empty lens folding cartridge. To guide and hold the lens folding cartridge a cylindrical opening is provided which encloses the back portion of the injecting nozzle. In addition a groove is provided holding the wing. Through the cylindrical opening a hook extends outward. If the lens folding cartridge is inserted into the device the hook extends through the injector nozzle into the front part of the half shell. Now the lens is put on the lens folding cartridge in such a way that the leading haptic can be caught by the hook and that the trailing haptic rests in the notch. The operating surgeon finds the device together with the lens folding cartridge and the lens in this condition and packed in a sterile packing. For use he takes the device from the packing and rotates the wing until it engages in the snap device. The lens is now folded. When the lens folding cartridge is pulled from the device the leading haptic is moved by the hook into the injector nozzle.

The present invention refers to a loading device for loading a lens folding cartridge with an intraocular lens.

For cataract operations it is now a standard procedure to insert artificial lenses, so-called intraocular lenses, into the capsular bag of the eye. In the past years so-called cartridges have been developed to insert intraocular lenses. The lens is loaded into the cartridge and then injected into the previously emptied capsular bag of the eye by means of an injector. Such cartridges and injectors are known from the U.S. Pat. No. 6,283,975 and WO03/045285.

The usual cartridges, as they are for example described in EP 1 023 880, comprise a loading channel with two half-shells connected by a film hinge. An intraocular lens can be placed on these half-shells. Wings are provided at the half-shells to facilitate closing of the cartridge. By closing the cartridge the lens is folded. In front of the loading channel is an injector nozzle through which the folded lens, after inserting the cartridge into an injector, can be injected.

Much skill of the operating surgeon is required for the correct insertion of the lens into the cartridge. Accordingly, in WO03/045285 a set is proposed in which the intraocular lens is already contained in tensionfree condition in the cartridge which, for folding the lens, has only to be closed. Until its use this cartridge is carried by a holder and remains in sterile condition in a packing. In case of a hydrophile lens, the lens is immersed in a liquid to protect it from drying. For the operation the cartridge together with the lens contained therein is removed from the packing, folded and inserted into an injector. After filling a visco-elastic liquid into the injection channel of the cartridge the system is ready for the injection of the intraocular lens into the capsular bag of the eye to be treated.

It has become evident that the handling of the described cartridges by the operating surgeon requires much skill because the cartridges are of small dimensions. In particular the handling of intraocular lenses with filamentary haptics is very delicate.

It is therefore an object of the present invention to show way and means to facilitate handling of the lens folding cartridges and in particular to prevent damage to the lens, especially to their haptics.

According to the invention a loading device for loading a lens folding cartridge with an intraocular lens is provided which is characterized by a support with a seat to receive the lens folding cartridge. Already the manufacturer of the lens can insert the lens in unfolded condition into this device into its optimal position. Accordingly, the operating surgeon or his assistant must only swivel a wing of the lens folding cartridge to fold the lens. This is easy to effect because the lens folding cartridge is located in a device which is larger than the lens folding cartridge. This substantially facilitates handling.

The loading device preferably comprises a support of plastic material. On this support a recess forms a seat for the lens folding cartridge. Preferably, the support comprises guide means for inserting the lens folding cartridge. The guide means may have a guide, e.g. an opening, guiding the injector nozzle. A second guide, e.g. a groove, may serve to guide the outer edge of a wing of the lens folding cartridge. After inserting the lens folding cartridge into the loading device the cartridge is held by these guides, but a wing remains swivelable.

Of particular advantage is a hook extending outward through the guide for the injector nozzle. When the lens folding cartridge is inserted into the device, the hook extends through the injector nozzle into the front part of the loading channel. On inserting a lens with filamentary haptics into the loading channel care is to be taken to bring the leading filamentary haptic into a position in which, on removal of the cartridge from the loading device, it is seized by the hook. Accordingly, when the operating surgeon or his assistant later on folds the lens folding cartridge and removes it from the loading device, the leading haptic is moved by the stationary hook into the injector nozzle and thereby straightened.

The loading device may comprise a locking member to keep the lens folding cartridge, after inserting an intraocular lens, in a partially open position. This ensures that the lens will not fall from the lens folding cartridge. On the other hand, no tension is exerted on the lens.

Preferably, the locking member is provided with a gripper for the trailing haptic to locate the trailing haptic, on unlocking, correctly for the injecting operation. For this purpose the locking element is swivelably located on an axis of the support. To permit turning the locking member may be designed as a manually operatable swivel arm. The gripper for the trailing haptic may be formed on this swivel arm. The locking member may comprise a stop to keep the lens folding cartridge in partially open position in which the lens is kept tensionfree. For use with a loading device as described before the lens folding cartridge comprises a loading channel consisting of two half shells connected together with a hinge, wings located on the half shells and an injector nozzle located at the head of the loading channel. This lens folding cartridge is characterized in that at the rear end of one of the half shells a notch is located to receive the trailing haptic. It would also be possible to have for the lens folding cartridge a functionally equivalent design comprising two film hinges, as for example shown in the U.S. Pat. No. 5,947,975.

The invention concerns also a set for implantation of an intraocular lens comprising a lens folding cartridge and characterized in that it further comprises a loading device for loading the lens folding cartridge. The set preferably comprises also an injector. The loading device with the lens folding device and the lens safely held tensionfree by the lens folding cartridge may be enclosed in a sterile packing.

Embodiments of the invention will now be described with reference to the drawing.

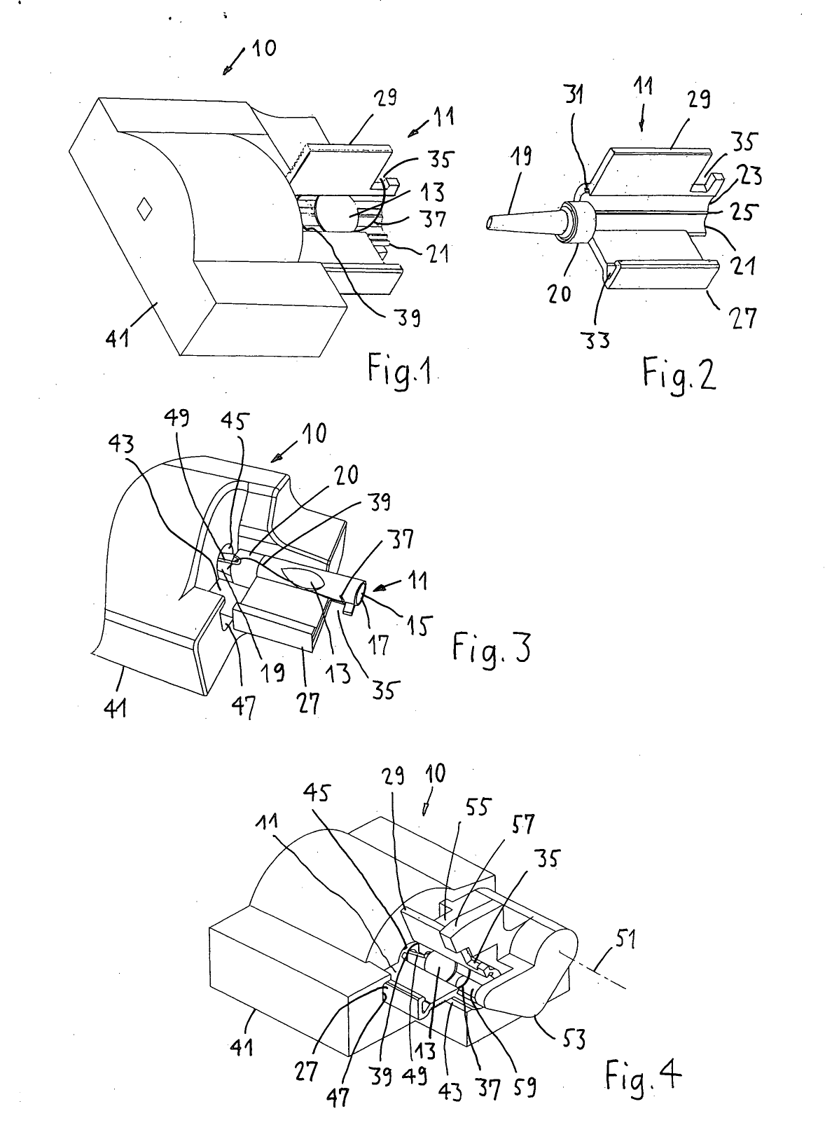

FIG. 1 shows a first embodiment of a loading device together with an open lens folding cartridge and an unfolded intraocular lens.

FIG. 2 shows a lens folding cartridge which is specially designed for use together with a loading device according to FIG. 1.

FIG. 3 shows a different view of the lens folding cartridge of FIG. 1 on occasion of the removal of the closed lens folding cartridge together with folded lens.

FIG. 4 shows a further embodiment of a loading device which is provided with a locking element.

FIGS. 1 and 3 show a loading device 10 together with a lens folding cartridge 11. Visible is also the intraocular lens 13. In FIG. 1 it is shown in unfolded condition and in FIG. 3 in folded condition.

The lens folding cartridge 11, best seen in FIG. 2, consists of preferably transparent plastic material and comprises a cylindrical loading channel 15 (FIG. 3) with an entrance opening 17 and an injector nozzle 19 connected in alignment with a loading channel 15. The lens folding cartridge 11 is foldable. The loading channel 15 comprises two cylindrical half shells 21, 23 connected together with a film hinge 25. Wings 27, 29 are provided at the half shells 21, 23. At the inside of each half shell 21, 23 is a longitudinal groove 31 (only one is visible). On inserting of the lens 13 into the cartridge 11 the peripheries of the lens 13 are engaged by the longitudinal grooves 31. By locating the longitudinal grooves 31 off the middle of the half shells 21, 23 the lens 13 will always be folded in direction of the film hinge 25 when the cartridge is folded. A snap device 33 is formed on a wing 27 so that after folding of the lens 13 the cartridge remains in closed position. The described design corresponds substantially to Prior Art, as for example described in WO03/045285. In contrast to the Prior Art, however, the cartridge 11 shown in FIG. 2 has a notch 35 to receive the trailing haptic 37 ot the lens 13. In the FIGS. 1 and 3 the leading haptic is designated with the reference numeral 39.

The loading device 10 comprises a support 41 consisting preferably of plastic material. A recess 43 (FIG. 3) in the support 41 serves as bed to receive the lens folding cartridge.

Guide means 45, 47 serve to insert the lens folding cartridge 11. The opening 45 guides the rear part 20 of the injector nozzle 19 and the groove 47 serves to guide the outer end of the wing 27 of the lens folding cartridge 11. In this way the lens folding cartridge 11 is firmly held in the loading device 10, but the wing 29 remains swivelable (FIG. 1). A hook 49 extends coaxially through the hole 45 in the interior of the support 41 and sticks out of this hole 45 (FIG. 3). This hook 49 has a purpose to place the leading haptic 39 correctly into the injector nozzle 19 when the closed lens folding cartridge 11 is removed from the loading device 10. In this way damage to the haptic 39 is prevented when the lens 13 is injected through the injector nozzle 19 into the capsular bag of the eye. When the loading device is used for lenses consisting of one piece, i.e. lenses without filamentary haptics, the hook 49 can be omitted.

The loading of an intraocular lens 13 into the lens folding cartridge is accomplished as follows:

The lens folding cartridge 11 is inserted into the loading device 10. After inserting the lens into the lens folding cartridge 11 the hook 49 (not visible in FIG. 1) extends through the injector nozzle 19 into the front region of the half shell 21. Then the loading device 10 can be held with one hand and the wing 29 tilted back with the other hand. With tweezers or another suitable instrument the lens 13, as shown in FIG. 1, is put on the cartridge 11. Care is taken that the leading haptic 39 can be gripped by the hook 49 and that the trailing haptic 37 is in the notch 35. This loading takes place at the site of manufacturing.

For the operation the operating surgeon removes in the sterile section the loading device 10 together with the cartridge and the still unfolded lens 13 from the sterile packing and rotates the wing 29 until it snaps into the snapping device 33. The lens 13 is now folded. On removing the lens folding cartridge 11 (FIG. 3) the leading haptic 39 is caught by the hook 49 and is moved into the injecting nozzle 19 thereby being elongated. This can be easily checked if the lens folding cartridge consists of transparent plastic material.

The loading device according to the embodiment of FIG. 4 is substantially of the same design as the loading device according to the FIGS. 1 and 3. Accordingly, for the same parts the same reference numerals are used and it can also be referred to the previous description. However, the loading device according to FIG. 4 has additionally a locking element in the form of a lever 53 tiltable around the axis 51. A stop 55 is provided at the locking element 53 to hold the lens folding cartridge 11 in partially open position. This is a position in which the lens 13 is prevented from falling out but is kept in tensionless condition. A further stop 57 holds the lens folding cartridge 11 in the axial position as shown. At the locking element 53 a gripper 59 is provided on which the trailing haptic 37 rests. If the locking element 53 is moved clockwise against the force of a detent device (not shown) the gripper 59 moves the trailing haptic 37 into the notch 35 as shown in FIG. 1. The lens folding cartridge 11 can now be closed by operating the wing 29 and, as shown in FIG. 3, removed.

Different modifications of the loading device 10 are possible without deviating from the inventive idea. For example, the locking element 53 may have a stud which in the position of FIG. 4 enters an opening on the support to prevent moving of the locking element 53. To unlock the locking element 53 may be pulled until it abuts on a stop and the stud is not any more engaging the opening.

Summerising the following can be stated:

The loading device 10 to load a lens folding cartridge with an intraocular lens 13 comprises a support 41 with a seat 43 to receive the still empty lens folding cartridge 11. To guide and hold the lens folding cartridge 11 a cylindrical opening 45 is provided which encloses the back portion 20 of the injecting nozzle 19. In addition a groove 47 is provided holding the wing 27. Through the cylindrical opening 45 a hook 49 extends outward. If the lens folding cartridge 11 is inserted into the device the hook 49 extends through the injector nozzle 19 into the front part of the half shell 21. Now the lens 13 is put on the lens folding cartridge 11 in such a way that the leading haptic 39 can be caught by the hook 49 and that the trailing haptic 37 rests in the notch 35. The operating surgeon finds the device 10 together with the lens folding cartridge 11 and the lens 13 in this condition and packed in a sterile packing. For use he takes the device 10 from the packing and rotates the wing 29 until it engages in the snap device 33. The lens 13 is now folded. When the lens folding cartridge 11 is pulled from the device 10 the leading haptic 39 is moved by the hook 49 into the injector nozzle 19.

1. Loading device for loading a lens folding cartridge with an intraocular lens, characterized by a support with a seat to receive the lens folding cartridge.

2. Loading device as claimed in claim 1, characterized in that the support comprises guide means for inserting the lens folding cartridge.

3. Loading device as claimed in claim 2, characterized in that the guide means comprise a guide, e.g. an opening, guiding the injector nozzle.

4. Loading device as claimed in claim 1, characterized in that it comprises a hook extending outward through the guide for the injector nozzle.

5. Loading device as claimed in claim 2, characterized in that the guide means comprise a guide, e.g. a groove, to guide the outer edge of a wing of the lens folding cartridge.

6. Loading device as claimed in claim 1, characterized in that it comprises a locking member to keep the lens folding cartridge, after insertion of an intraocular lens, in a partially open position.

7. Loading device as claimed in claim 6, characterized in that the locking member comprises a gripper for the trailing haptic to position the trailing haptic, on delocking, correctly for the injecting operation.

8. Loading device as claimed in claim 6, characterized in that the locking member is located on a axis of the support.

9. Loading device as claimed in claim 6, characterized in that the locking member is a manually operatable swivel arm.

10. Loading device as claimed in claim 6, characterized in that the locking member comprises a stop to keep the lens folding cartridge in a partly open position.

11. Lens folding cartridge for use with a loading device as claimed in claim 1, with a loading channel comprising two half shells, wings located on the half shells and a injector nozzle located at the head of the loading channel, characterized in that at the rear of one of the half shells notch is located to receive the trailing haptic.

12. Set for the implantation of an intraocular lens comprising a lens folding cartridge, characterized in that it further comprises a loading device according to claim 1 to load the lens folding cartridge.

13. Set as claimed in claim 12, characterized in that it further comprises an injector into which the lens folding cartridge can be inserted.

14. Set as claimed in claim 12, characterized in that the loading device with the lens folding cartridge and the lens safely held by the lens folding cartridge, but unfolded, are stored in a sterile packing.
